(19) United States
(12) Patent Application Publication
(10) Pub. No.: US 2026/0146986 A1
(43) Pub. Date: May 28, 2026

(54) ANALYSIS OF NON-SIEVED DRILLING CUTTINGS

(71) Applicant: Schlumberger Technology Corporation, Sugar Land, TX (US)

(72) Inventors: Simone Di Santo, Dammam (SA); Tetsushi Yamada, Cambridge, MA (US)

(21) Appl. No.: 19/387,063

(22) Filed: Nov. 12, 2025

Related U.S. Application Data
(60) Provisional application No. 63/723,885, filed on Nov. 22, 2024.

(51) Int. Cl. G01N 33/24; G01N 1/08; G01N 21/64; G06T 7/40; G06T 7/50; G06V 10/143; G06V 10/26; G06V 10/40

(52) U.S. Cl. CPC G01N 33/24; G01N 1/08; G01N 21/6456; G06T 7/40; G06T 7/50; G06V 10/143; G06V 10/26; G06V 10/40; G06T 2207/10016; G06T 2207/10064; G06T 2207/10152; G06T 2207/30181

(57) ABSTRACT

A method for analyzing drilling cuttings includes extracting a sample of drilling cuttings from a subterranean formation, the sample including consolidated particles and unconsolidated material. The method includes photographing the sample to produce a photograph, performing an image analysis on the photograph to identify segments of the photograph visualizing the unconsolidated material and excluding visualization of the consolidated particles, and analyzing the segments by performing at least one of a spectral measurement, a texture analysis, a grain size distribution analysis, or a reservoir parameter estimation of the segments. The method enables extraction of geological information from both consolidated and unconsolidated fractions of drilling cuttings samples that would otherwise be discarded in conventional sieving processes, thereby preserving subsurface information including grain size, mineral composition, and other parameters for comprehensive geological characterization of subterranean formations.

### CROSS-REFERENCE TO RELATED APPLICATIONS

This application claims priority to U.S. Provisional Application No. 63/723,885, filed on 22 Nov. 2024, which is hereby incorporated by reference in its entirety.

### BACKGROUND

Automated lithology analysis focuses on automating the examination of drilling cuttings for geological characterization in oil and gas operations. Traditional workflows employ Object Based Image Analysis (OBIA) methodologies that leverage the identification of regions of interest within images, where these regions are characterized with parameters that can be recognized as objects. When applied to rock particles for lithological characterization, this method is termed Lithological Object Based Analysis (LiOBIA). Current standard procedures involve sieving drilling cuttings to remove finer components, typically with a lower threshold size limit, which allows observation of particles representative of rock in its aggregated form and reveals natural texture and other geological and mineralogical aspects of drilled formations. However, in many formations and drilling conditions, poorly cemented lithologies come to the surface disaggregated into their constituent components smaller than the threshold, particularly within clastic formations where poorly consolidated sandstones result in rocks arriving at the surface as loose materials. The loose fraction from such formations is often discharged and not utilized in automated lithology workflows, resulting in the loss of subsurface information including grain size, mineral composition, and other parameters that could provide valuable geological insights.

### SUMMARY

According to an aspect of the present disclosure, a method for analyzing drilling cuttings is provided. The method includes extracting a sample of drilling cuttings from a subterranean formation, the sample including consolidated particles and unconsolidated material. The method includes photographing the sample to produce a photograph. The method includes performing an image analysis on the photograph to identify segments of the photograph visualizing the unconsolidated material and excluding visualization of the consolidated particles. The method includes analyzing the segments by performing at least one of a spectral measurement, a texture analysis, a grain size distribution analysis, or a reservoir parameter estimation of the segments.

According to other aspects of the present disclosure, the method may include one or more of the following features. The segments may be first segments, and performing the image analysis may include identifying second segments of the photograph visualizing the consolidated particles. The method may further include analyzing the second segments. Analyzing the second segments may include performing at least one of a shape measurement, a texture measurement, a spectral measurement, or a feature extraction of the second segments. Extracting the sample from the subterranean formation may include drilling into a poorly cemented lithology. The poorly cemented lithology may include at least one of sandstone or carbonate. The unconsolidated material may include loose sand from sandstone.

According to another aspect of the present disclosure, a method for analyzing drilling cuttings is provided. The method includes extracting, from a geological formation, a sample of non-sieved drilling cuttings including consolidated rock particles and unconsolidated loose material. The method includes acquiring, via a camera, a photograph of the sample. The method includes performing image segmentation on the photograph to identify the consolidated rock particles as segmented instances. The method includes partitioning the photograph into a plurality of patches. The method includes extracting a subset of patches of the plurality of patches, the subset of patches being non-overlapping with the segmented instances. The method includes analyzing the subset of patches via a first processing pipeline to obtain first geological information of the geological formation from the unconsolidated loose material. The method includes analyzing the segmented instances through a second processing pipeline to obtain second geological information of the geological formation from the consolidated rock particles.

According to other aspects of the present disclosure, the method may include one or more of the following features. Each patch of the plurality of patches may be non-overlapping with adjacent patches of the plurality of patches. The method may further include applying a first minimum threshold test to the subset of patches based on a first total number of individual patches of the subset of patches or a first area of the photograph covered by the subset of patches, and applying a second minimum threshold test to the segmented instances based on a second total number of the segmented instances or a second area of the photograph covered by the segmented instances. The method may further include discharging the subset of patches that fall below the first minimum threshold, and discharging the segmented instances that fall below the second minimum threshold. Analyzing the subset of patches may include performing at least one of a spectral measurement, a texture analysis, a grain size distribution analysis, or a reservoir parameter estimation. Analyzing the segmented instances may include performing at least one of a shape measurement, a texture measurement, a spectral measurement, or a feature extraction. The unconsolidated loose material may include disaggregated components from a poorly cemented lithology. The disaggregated components from the poorly cemented lithology may include loose sand from poorly consolidated sandstone or carbonate (e.g., grainstones). Acquiring the photograph may include taking a first photograph under visible white light illumination and a second photograph under UV light illumination. The UV light illumination may include at least one of short UV light illumination and long UV light illumination to produce a plurality of fluorescence colors in minerals present in the sample. The photograph may be acquired when the sample is on a tray having a contrast color background including at least one of magenta, blue, or green.

According to another aspect of the present disclosure, a method for analyzing a geological formation is provided. The method includes extracting a sample of drilling cuttings from the subterranean formation, the sample including consolidated particles and unconsolidated material. The method includes photographing the sample to produce a photograph. The method includes performing an image analysis on the photograph to identify first segments of the photograph visualizing the unconsolidated material and second segments of the photograph visualizing the consolidated particles. The method includes analyzing the first segments to obtain first information of the geological formation by performing at least one of a spectral measurement, a texture analysis, a grain size distribution analysis, or a reservoir parameter estimation of the first segments. The method includes analyzing the second segments to obtain second information of the geological formation by performing at least one of a shape measurement, a texture measurement, a spectral measurement, or a feature extraction of the second segments.

According to other aspects of the present disclosure, the method may include one or more of the following features. The geological formation may include a poorly cemented lithology. The unconsolidated material may include loose sand from the poorly cemented lithology. The consolidated particles may include rock particles from the poorly cemented lithology.

### BRIEF DESCRIPTION OF FIGURES

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 illustrates a wellsite system for drilling operations, according to aspects of the present disclosure;

FIG. 2 illustrates a flowchart of a method for analyzing drilling cuttings, according to aspects of the present disclosure;

FIG. 3 illustrates a workflow diagram for analyzing drilling cuttings, according to aspects of the present disclosure;

FIG. 4 shows an example of input and output of image segmentation for rock particles, according to aspects of the present disclosure;

FIG. 5 shows a comparison between white-light and UV-light illuminated photographs with processing paths, according to aspects of the present disclosure;

FIG. 6 illustrates instance segmentation of rock particles in a non-sieved drilling cutting image, according to aspects of the present disclosure;

FIG. 7 illustrates a workflow of image segmentation with parallel processing paths, according to aspects of the present disclosure;

FIG. 8 illustrates a schematic representation of a workflow for image segmentation and partitioning, according to aspects of the present disclosure; and

FIG. 9 illustrates extracted consolidated and unconsolidated objects from a drilling cuttings analysis, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Embodiments of the present disclosure relate to methods for analyzing drilling cuttings obtained from subterranean formations. During drilling operations, rock samples may be brought to the surface in various forms, including consolidated rock particles and unconsolidated material. Conventional analysis techniques may focus on consolidated rock particles while discarding unconsolidated material, potentially resulting in loss of geological information.

Methods for analyzing drilling cuttings described herein may enable extraction of geological information from both consolidated and unconsolidated fractions of a sample. The methods may involve photographing a sample containing both consolidated particles and unconsolidated material, followed by image analysis to identify different segments of the photograph corresponding to these different material types. The segments may then be analyzed to obtain geological information about the formation from which the sample was extracted.

In some embodiments, a method for analyzing drilling cuttings may involve extracting a sample from a geological formation, where the sample includes non-sieved drilling cuttings containing both consolidated rock particles and unconsolidated loose material. The method may include acquiring photographs of the sample and performing image segmentation to identify and separately analyze the different material types present in the sample.

A method for analyzing a geological formation may utilize drilling cuttings samples to obtain information about subsurface formations. The method may involve photographing samples containing both consolidated particles and unconsolidated material, performing image analysis to identify segments corresponding to each material type, and analyzing these segments to extract geological information about the formation. Such methods may enable more comprehensive analysis of drilling cuttings by utilizing information from both consolidated and unconsolidated fractions that may be present in samples obtained during drilling operations.

During or after the analysis described, the near-real-time information gathered from the analysis an inform one or more changes in drilling operations. For example, the analysis and resulting information may be used to advise regarding changes in reservoir quality that enable informed drilling decisions even when logging while drilling is limited or absent. Also, for example, the methods and analysis described herein may collect continuous data that are normally available only in lab single-point measurements, for example grain size distribution. Another technical advantage of the methods and systems described herein includes post-mortem applications where cuttings archives can be used to extract the information when no log was performed, and a well was already drilled.

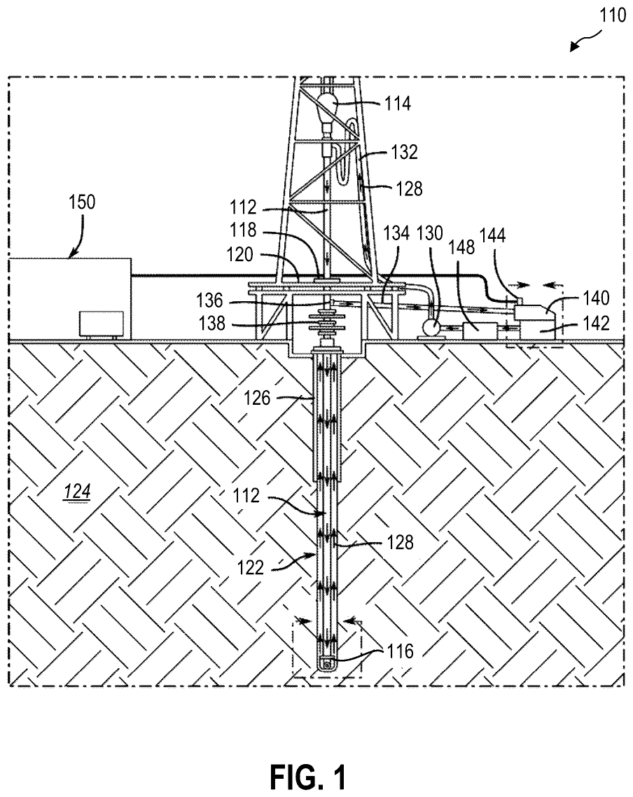

Referring to FIG. 1, a drilling operation 110 may be configured to extract samples from subterranean formations for geological analysis. The drilling operation 110 may include a drill string 112 that extends downward into a borehole 122 formed in an earth formation 124. A traveling block 114 may be positioned above the drill string 112 to facilitate movement of drilling equipment during operations.

A drill bit 116 may be located at the bottom end of the drill string 112 to cut through the earth formation 124 and create the borehole 122. The drill bit 116 may rotate to crush rock material, generating drilling cuttings that contain geological information about the earth formation 124. A rotary table 118 may be positioned on a driller floor 120 to provide rotational motion to the drill string 112 and the drill bit 116.

The drilling operation 110 may include a casing 126 that lines portions of the borehole 122 to provide structural support and prevent collapse of the borehole walls. Drilling fluid 128 may be circulated through the system to facilitate drilling operations and transport drilling cuttings to the surface. A mud pump 130 may be connected to the system via a mud line 132 to circulate the drilling fluid 128 downward through the drill string 112.

The drilling fluid 128 may flow back to the surface through a return flow line 134 after reaching the bottom of the borehole 122. The return flow line 134 may carry the drilling fluid 128 along with drilling cuttings that have been generated by the drill bit 116. A bell nipple 136 may be positioned at the top of the borehole 122 to direct the returning drilling fluid 128 and cuttings into the return flow line 134.

A blowout preventer 138 may be installed to provide safety control during drilling operations by preventing uncontrolled release of formation fluids. The drilling fluid 128 and cuttings may be directed to a shale shaker 140 that separates the drilling cuttings from the drilling fluid 128. The shale shaker 140 may use vibrating screens to separate solid particles from the liquid drilling fluid 128.

A shaker pit 142 may be positioned below the shale shaker 140 to collect the separated drilling cuttings for further processing and analysis. The drilling fluid 128 may be processed through additional equipment including a gas trap 144 that removes gas from the drilling fluid 128 before the fluid is stored in a mud pit 148 for reuse in the drilling operation 110.

A control system 150 may monitor and control various aspects of the drilling operation 110, including the circulation of drilling fluid 128 and the collection of drilling cuttings. The drilling cuttings collected from the shaker pit 142 may contain both consolidated rock particles and unconsolidated material that can provide geological information about the earth formation 124. These drilling cuttings may be prepared for analysis using the methods described herein to extract comprehensive geological data from both consolidated and unconsolidated fractions of the sample.

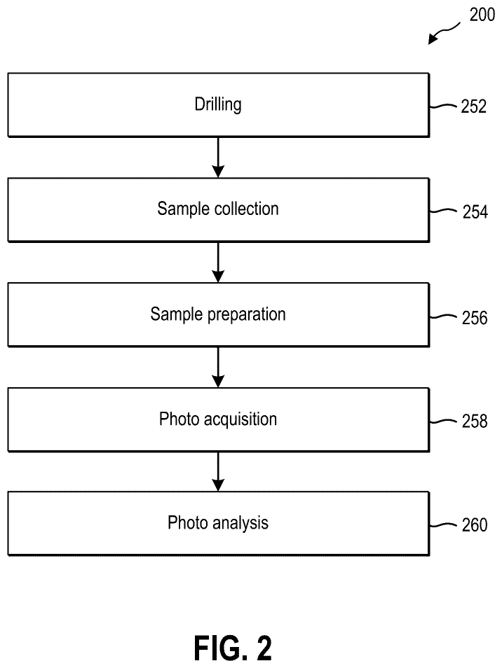

Referring to FIG. 2, a workflow 200 may illustrate an overall method for analyzing drilling cuttings obtained from subterranean formations. The workflow 200 may include a sequence of steps that enable comprehensive analysis of both consolidated particles and unconsolidated material present in drilling cuttings samples. The workflow 200 may be implemented to extract geological information that might otherwise be lost when conventional analysis techniques focus solely on consolidated rock particles.

At block 252, the workflow 200 may begin with a drilling step that involves extracting a sample of drilling cuttings from a subterranean formation. The sample may include consolidated particles and unconsolidated material that are generated during drilling operations when the drill bit 116 cuts through the earth formation 124. The drilling step may produce cuttings that contain geological information about the subsurface formation being drilled. In some embodiments, the sample may include disaggregated components from poorly cemented lithologies that come to the surface in both consolidated and unconsolidated forms.

At block 254, the workflow 200 may proceed to a sample collection step where the drilling cuttings are gathered from the drilling operation 110. The sample collection may involve retrieving the drilling cuttings from the shale shaker 140 after the cuttings have been separated from the drilling fluid 128. The collected sample may contain both consolidated rock particles that maintain their structural integrity and unconsolidated material such as loose grains or crystals that have become disaggregated during the drilling process.

At block 256, the workflow 200 may continue with a sample preparation step that prepares the collected drilling cuttings for photographic analysis. The sample preparation may involve drying the collected rock particles using an oven or other drying techniques to remove moisture that could interfere with subsequent analysis. In some embodiments, the sample preparation may include placing the dried particles on a tray with a vivid contrast background color such as magenta, blue, or green to facilitate image segmentation processes. The sample preparation step may maintain both consolidated particles and unconsolidated material in the sample without sieving, thereby preserving geological information that would otherwise be discarded.

At block 258, the workflow 200 may proceed to a photo acquisition step where photographs of the prepared sample are captured. The photo acquisition may involve taking photographs under visible white light illumination and under UV light illumination to reveal different characteristics of the sample materials. The photographs may be acquired using a camera system that captures images of the sample containing both consolidated particles and unconsolidated material in their prepared arrangement on the tray. The photo acquisition step may produce digital images that serve as input for subsequent image analysis processes.

At block 260, the workflow 200 may conclude with a photo analysis step that extracts geological information from the acquired photographs. The photo analysis may involve performing image analysis on the photographs to identify segments corresponding to unconsolidated material and segments corresponding to consolidated particles. The analysis may include processing the identified segments to obtain geological information about the subterranean formation through various measurement techniques such as spectral measurements, texture analysis, grain size distribution analysis, or reservoir parameter estimation. The photo analysis step may enable extraction of comprehensive geological data from both consolidated and unconsolidated fractions of the drilling cuttings sample.

Sample preparation procedures may involve several steps to prepare drilling cuttings for photographic analysis. The drilling cuttings sample may be dried using an oven or other drying techniques to remove moisture that could interfere with subsequent image analysis processes. In some embodiments, the drilling cuttings may be sieved using two meshes with sizes ranging from 0.25 mm to 3 mm for lower and upper bounds respectively, though the method may also accommodate non-sieved drilling cuttings including consolidated rock particles and unconsolidated loose material. These size ranges are exemplary only and not meant to be limiting. The size of the meshes may dictate a threshold size for rock particle analysis, where particles below the threshold may be considered loose, unconsolidated material. The sample may be placed on a tray having a contrast background color, for example magenta, blue, or green background color to facilitate image segmentation by providing contrast with the natural colors of rock materials. During sample preparation, any piled particles may be sparsely distributed to other areas in the tray to prevent overlapping that could interfere with individual particle identification during image analysis.

Photographing the sample to produce a photograph may involve acquiring images under different illumination conditions to reveal various characteristics of the sample materials. In at least one example, a multi or hyper-spectral sensor may be used to acquire a photograph of the sample. A camera may be used to acquire a photograph of the sample, where the camera may be enclosed within a box during the acquisition workflow to provide controlled lighting conditions and reduce external interference. The method may include taking a first photograph under visible white light illumination and a second photograph under UV light illumination to capture different properties of the minerals present in the sample. The UV light illumination may include at least one of short UV light illumination and long UV light illumination to produce a plurality of fluorescence colors in minerals present in the sample, enabling enhanced differentiation between different mineral types that may appear similar under visible light.

The photograph acquisition process may capture the sample in the exact same position across different illumination conditions, ensuring that subsequent image analysis can correlate features between different photographs of the same sample. The photographs may be taken using different sensors that are sensible to specific electromagnetic frequency ranges in the form of multispectral or hyperspectral imaging to provide additional spectral information about the sample materials. The photograph sequence may be automatically performed during the acquisition workflow, reducing manual intervention and ensuring consistent imaging conditions across multiple samples. The resulting set of photographs may provide comprehensive visual data for both consolidated particles and unconsolidated material present in the drilling cuttings sample.

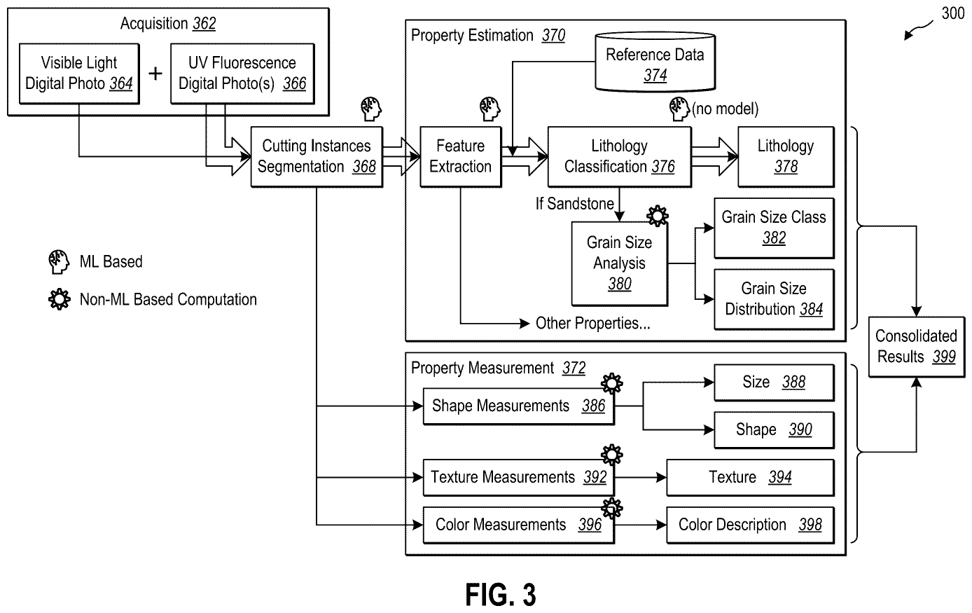

Referring to FIG. 3, a workflow 300 may illustrate a detailed analysis process for extracting geological information from drilling cuttings samples. The workflow 300 may include multiple computational components that process both consolidated rock particles and unconsolidated material to obtain comprehensive geological data about subterranean formations. The workflow 300 may integrate various measurement techniques and analysis methods to characterize different properties of the drilling cuttings sample. each block of the workflow 300 shown in FIG. 3 may include an icon indicating a process step which may be carried out by non-machine learning based computation and those that may be carried out using machine-learning based computations. These blocks and indications of machine-learning steps are exemplary only and not meant to be limiting. Other embodiments of the workflow 300 may include other scenarios where machine-learning computations is or is not used.

The workflow 300 may include an acquisition workflow 362 that handles the initial processing of input data from the drilling cuttings sample. The acquisition workflow 362 may receive input from visible light digital photographs at block 364 and UV fluorescence digital photographs at block 366 that have been captured during the photograph acquisition process.

At block 368, the outlining instance segmentation may create precise delineations around each identified object from the photographs to enable accurate measurements and analysis of individual components within the sample. At block 368, the acquisition workflow 362 may complete the initial processing steps and provide segmented data to subsequent analysis components within the workflow 300.

The workflow 300 may include a property estimation workflow 370 that processes the segmented data to estimate various geological properties of the sample materials. The property estimation workflow 370 may utilize both machine learning-based and non-machine learning-based computational approaches to analyze the characteristics of consolidated particles and unconsolidated material. The property estimation workflow 370 may generate quantitative estimates of geological properties that can be used to characterize the subterranean formation from which the sample was extracted.

A property measurement workflow 372 may operate in parallel with the property estimation workflow 370 to perform direct measurements on the segmented objects identified during the acquisition workflow 362. At block 374, reference data may be used as an input for the machine-learning computations within the property estimation workflow 370.

At block 376, the property measurement workflow 372 may estimate lithology classifications of the sample and determine lithology at 378. Also, after lithology classification at block 376, grain size analysis of the sample can occur as shown at block 380. After grain size analysis at block 380, the grain size class can be estimated at block 382 and the grain size distribution can be estimated at block 384.

With reference to the property measurement workflow 372, after cutting instances segmentation at block 368, the sample can undergo shape measurements at block 386, texture measurements at block 392, and spectral measurements at block 396. The spectral measurements may include multi or hyper-spectral measurements in some images. After shape measurements at block 386, the size (at block 388) and shape (at block 390) of the consolidated and unconsolidated material within the sample can be measured. After texture measurements at block 392, the texture can be determined at block 394. Likewise, after spectral measurements at block 396, the color description can be determined at block 398. The results from the property estimation workflow 370, including the lithology at block 378, the grain size class at block 382, and the grain size distribution at block 384, may be combined with the results from the property measurement workflow 372, including the size at block 388, the shape at block 390, the texture at block 394, and the color description at block 398, into consolidated results at block 399.

The final, consolidated results at block 399 of the workflow 300 may complete the analysis process by generating final results that characterize the geological properties of the subterranean formation. The workflow 300 may utilize both ML-based and non-ML based computational elements throughout the processing chain to ensure robust analysis of the drilling cuttings sample. The ML-based computations may employ machine learning algorithms to identify patterns and classify materials, while non-ML based computations may perform direct measurements and calculations based on established geological principles and reference data.

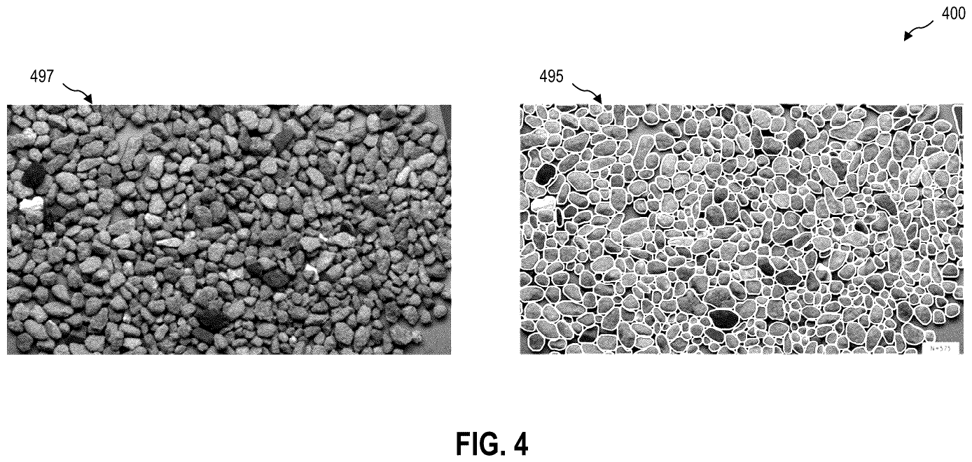

Referring to FIG. 4, the fundamental concept of Object Based Image Analysis may involve identifying relevant regions of interest within photographs 400 to isolate specific objects for detailed analysis. A photograph 497 may show rock particles distributed against a background, where image segmentation processes may distinguish between individual particle instances and background areas. The image segmentation may produce a segmented photograph 495 that identifies and delineates individual consolidated rock particles as segmented instances while excluding visualization of background areas and unconsolidated material. The segmentation process may enable performing an image analysis on the photograph to identify segments of the photograph visualizing the unconsolidated material and excluding visualization of the consolidated particles, or alternatively to identify first segments of the photograph visualizing the unconsolidated material and second segments of the photograph visualizing the consolidated particles. The regions of interest identified during segmentation may be propagated from white light photographs to UV light photographs or other band-specific images to maintain consistent object identification across different illumination conditions, and the image segmentation may be performed on combined multichannel images rather than individual photographs to utilize spectral information from multiple imaging sources simultaneously.

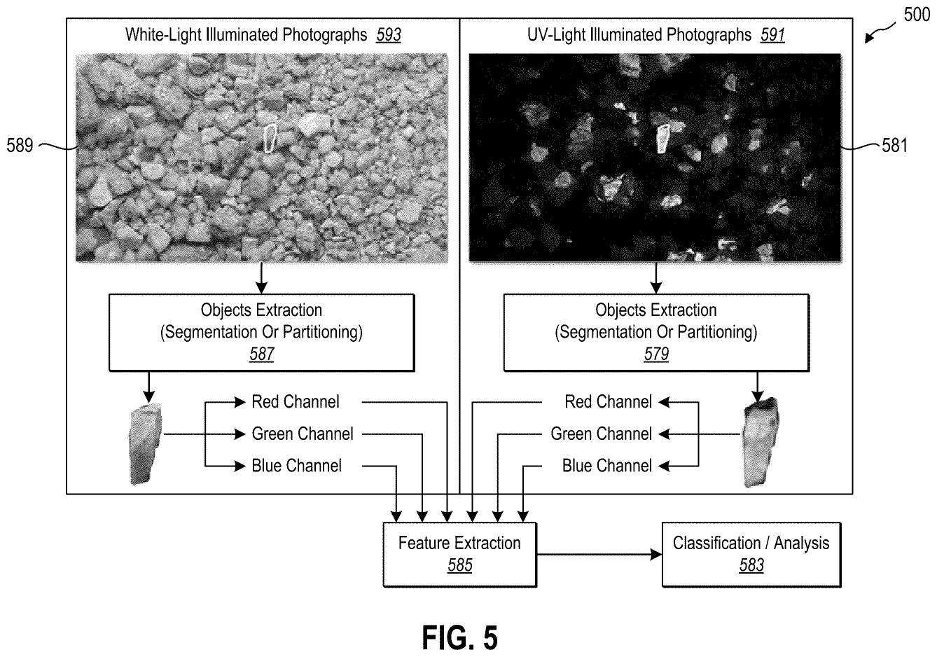

Referring to FIG. 5, a workflow 500 may illustrate the comparison between white light and UV light illuminated photographs for analyzing drilling cuttings samples, and the use of both photographs to accomplish feature extraction and classification of samples. The workflow 500 may demonstrate how different illumination methods can reveal distinct characteristics of the same rock sample, with UV illumination showing fluorescent properties that may not be visible under white light. Different minerals may emit different fluorescence colors under UV illumination, enabling enhanced differentiation between mineral types that may appear similar in color and texture when illuminated with visible light alone.

The workflow 500 may include parallel processing paths that handle both illumination types to maximize the geological information extracted from drilling cuttings samples. A white light workflow 593 may process data obtained from visible light illumination, while a UV-light workflow 591 may process data obtained from ultraviolet illumination. The parallel processing approach may enable comprehensive analysis by combining spectral information from both illumination conditions to improve rock-mineral differentiation and classification accuracy.

A white light photograph 589 may be captured during the visible light illumination phase of the photograph acquisition process. The white light photograph 589 may show the natural colors and textures of consolidated rock particles and unconsolidated material as they appear under standard visible light conditions. At block 587, the white light workflow 593 may perform object extraction through segmentation or partitioning processes to identify individual rock particles and unconsolidated material segments within the white light photograph 589.

At block 585, the white light workflow 593 may perform RGB channel separation to isolate red, green, and blue color components from the white light photograph 589. The RGB channel separation may enable detailed analysis of color characteristics and spectral properties of the sample materials under visible light illumination. The white light workflow 593 may process the separated RGB channels to extract features and measurements that characterize the visible light properties of the consolidated particles and unconsolidated material.

A UV-light photograph 581 may be captured during the ultraviolet illumination phase to reveal fluorescent properties of minerals present in the drilling cuttings sample. The UV-light photograph 581 may display fluorescence colors that are not visible under standard white light illumination, providing additional spectral information for mineral identification and classification. At block 579, the UV-light workflow 591 may perform object extraction processes similar to those performed in the white light workflow 593, but applied to the fluorescent characteristics revealed in the UV-light photograph 581.

The UV-light workflow 591 may also include RGB channel separation processes that isolate the fluorescent color components captured in the UV-light photograph 581. The fluorescent RGB channels may contribute to improved rock-mineral differentiation by revealing mineral-specific fluorescence patterns that are not apparent in visible light imaging. The UV-light workflow 591 may process the fluorescent RGB channels to extract features that complement the information obtained from the white light workflow 593.

The workflow 500 may combine extracted features from both the white light workflow 593 and the UV-light workflow 591 to perform comprehensive feature extraction (at block 585) and classification analysis (at block 583). The combination of visible light and fluorescent spectral information may enable more accurate identification of mineral types and geological properties than either illumination method alone. The integrated analysis may utilize the complementary information from both illumination conditions to enhance the overall accuracy of lithological characterization and geological formation analysis.

The fluorescence colors observed in the UV-light photograph 581 may vary depending on the specific minerals present in the drilling cuttings sample and the wavelength of UV illumination used during photograph acquisition. Short UV light illumination and long UV light illumination may produce different fluorescence responses in the same minerals, providing additional spectral discrimination capabilities. The workflow 500 may accommodate multiple UV wavelengths to maximize the fluorescent information available for mineral identification and classification processes.

The parallel processing approach implemented in the workflow 500 may ensure that both visible light and fluorescent characteristics are preserved and utilized during the analysis of drilling cuttings samples. The white light workflow 593 may provide information about natural mineral colors, textures, and morphological features, while the UV-light workflow 591 may reveal fluorescent properties that indicate specific mineral compositions and crystalline structures. The combination of these complementary data sources may result in more comprehensive and accurate geological characterization of the subterranean formations from which the drilling cuttings samples were extracted.

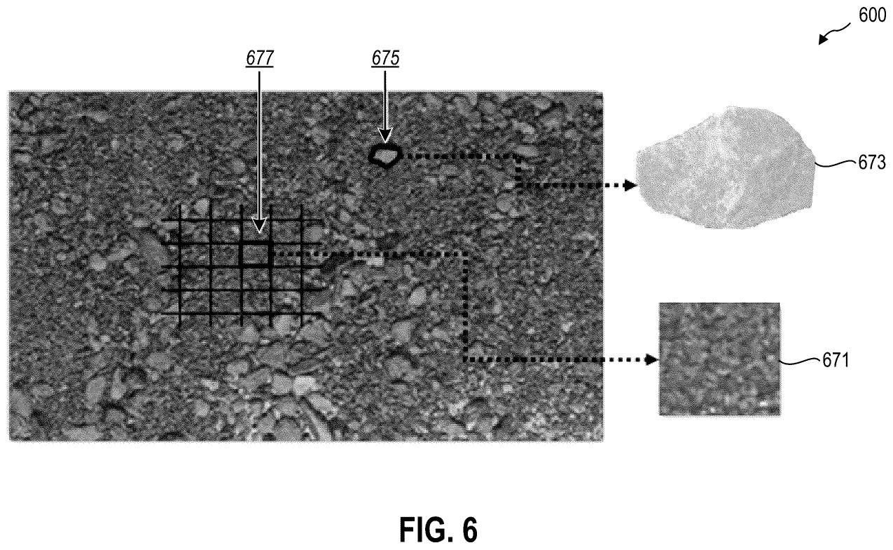

Referring to FIG. 6, a workflow 600 may illustrate a hybrid segmentation approach that combines instance segmentation of consolidated rock particles with patch partitioning of unconsolidated material in non-sieved drilling cuttings samples. The workflow 600 may enable comprehensive analysis of both consolidated and unconsolidated fractions present in drilling cuttings by applying different image processing techniques to different material types within the same photograph. The hybrid approach may maximize the geological information extracted from drilling cuttings samples by preserving and analyzing both consolidated rock particles and loose material that would otherwise be discarded in conventional sieving processes.

The workflow 600 may implement a segmentation 675 process that identifies consolidated rock particles as individual instances within the photograph. The segmentation 675 may distinguish between consolidated particles that maintain their structural integrity and unconsolidated material that appears as loose grains or disaggregated components in the sample. An image instance (rock particle) 673 may represent a consolidated rock particle that has been identified and delineated during the segmentation 675 process. The image instance (rock particle) 673 may maintain its natural irregular shape and boundaries as determined by the instance segmentation algorithms applied to the photograph.

The workflow 600 may simultaneously implement a partitioning 677 process that divides the photograph into uniform patches to capture unconsolidated material present in areas between the segmented rock particles. The partitioning 677 may create a systematic grid-based division of the photograph that enables analysis of loose material that cannot be effectively processed using instance segmentation techniques. An image patch (loose grains) 671 may represent a uniform rectangular or square portion of the photograph that contains unconsolidated material such as loose sand, disaggregated crystals, or other fine-grained components.

The method may involve partitioning the photograph into a plurality of patches through the partitioning 677 process. The plurality of patches may be created with predefined dimensions and may cover the entire photograph in a systematic manner. In some embodiments, each patch of the plurality of patches may be non-overlapping with adjacent patches of the plurality of patches, creating a regular grid pattern that divides the photograph into discrete analysis units. Alternatively, the patches in the partitioning process may be extracted with or without overlapping between adjacent patches, allowing for flexible patch extraction strategies depending on the specific analysis requirements and the characteristics of the unconsolidated material present in the sample.

The method may include extracting a subset of patches of the plurality of patches, where the subset of patches may be non-overlapping with the segmented instances identified during the segmentation 675 process. The extraction process may identify patches that contain primarily unconsolidated material by excluding patches that overlap with the image instance (rock particle) 673 or other consolidated particles identified during instance segmentation. The subset of patches may represent areas of the photograph that contain loose grains, disaggregated mineral components, or other unconsolidated material that can provide geological information about the subterranean formation.

The workflow 600 may ensure that the image patch (loose grains) 671 extracted during the partitioning 677 process does not overlap with the image instance (rock particle) 673 identified during the segmentation 675 process. This separation may enable independent analysis of consolidated and unconsolidated material types using appropriate processing techniques for each material category. The non-overlapping extraction approach may prevent double-counting of material components and ensure that each portion of the photograph is assigned to the most appropriate analysis pathway based on the material type present in that region.

The hybrid segmentation approach may enable performing an image analysis on the photograph to identify first segments of the photograph visualizing the unconsolidated material and second segments of the photograph visualizing the consolidated particles. The first segments may correspond to the image patch (loose grains) 671 extracted through the partitioning 677 process, while the second segments may correspond to the image instance (rock particle) 673 identified through the segmentation 675 process. The identification of both segment types within the same photograph may enable comprehensive analysis of the complete drilling cuttings sample without loss of geological information from either material type.

The workflow 600 may process the image patch (loose grains) 671 and the image instance (rock particle) 673 through separate analysis pipelines that are optimized for the characteristics of each material type. The image patch (loose grains) 671 may be analyzed using techniques appropriate for loose material such as grain size distribution analysis, texture analysis of disaggregated components, and spectral measurements of individual mineral grains. The image instance (rock particle) 673 may be analyzed using techniques appropriate for consolidated particles such as shape measurements, morphological analysis, and structural characterization of intact rock fragments.

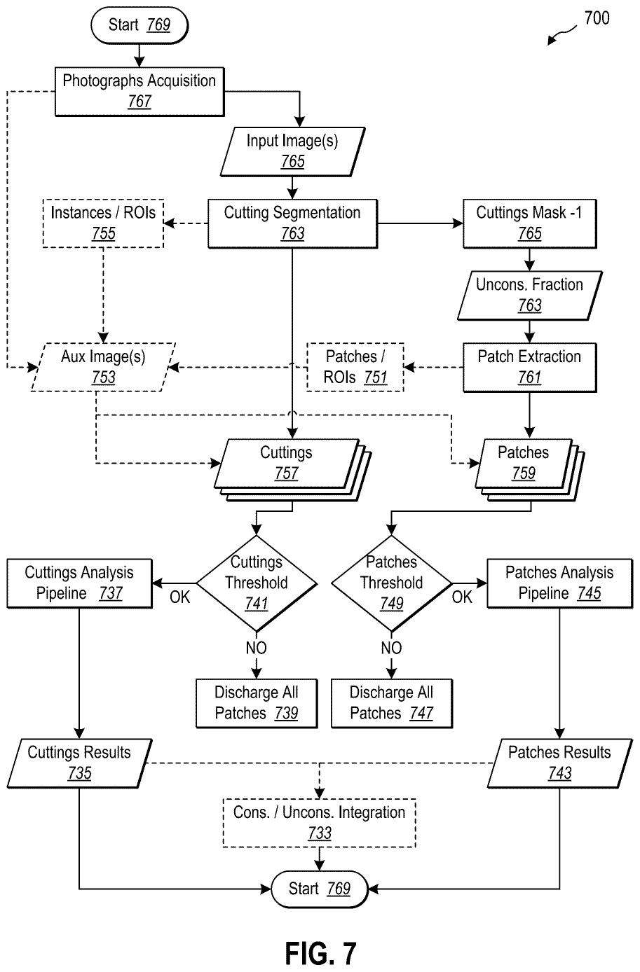

Referring to FIG. 7, a workflow 700 may illustrate a detailed segmentation process that enables comprehensive analysis of both consolidated rock particles and unconsolidated material present in non-sieved drilling cuttings samples. The workflow 700 may implement a systematic approach to image processing that includes photograph acquisition, segmentation of consolidated particles, extraction of unconsolidated material patches, threshold testing procedures, and routing of processed data to appropriate analysis pipelines. The workflow 700 may ensure that geological information from both material types is preserved and analyzed while maintaining quality control through threshold-based filtering mechanisms.

Using block 769 as a starting point, the first step of the workflow 700 may include photograph acquisition at block 767. At block 765, the images may be input for analysis and cutting segmentation at block 763. In one embodiment, after cutting segmentation at block 763, the cuttings mask may be performed at block 765 to determine a fraction of unconsolidated material in the photographed sample at block 763, after which patch extraction may be performed at block 761. Block 759 represents the various extracted patches. Also, after cutting segmentation at block 763, block 757 represents the stored or identified cuttings from the segmented photograph of the sample.

The dotted lines and blocks shown in FIG. 7 may indicate optional or alternative steps of the workflow 700. For example, at block 755, after the cutting segmentation at block 763, regions of interest (ROIs) and instances may be identified and auxiliary images of the same may be produced and/or stored at block 753. Similarly, after patch extraction at block 761, ROIs and patches may be identified at block 751 and used to identify/store the auxiliary images at block 753, which can then be input as part of the cuttings and patches at blocks 757 and 759, respectively.

A cuttings threshold at block 757 and a patches threshold at block 759 may be established and analyzed. In at least one embodiment, the cutting threshold may utilize various criteria to evaluate the adequacy of material representation in the drilling cuttings sample. The first minimum threshold applied to patches may be based on a minimum number of patches, such as requiring at least 10 individual patches, or a minimum area coverage, such as requiring patches to occupy at least 2% of the total photograph area in pixels. Similarly, the second minimum threshold applied to segmented instances may be based on a minimum number of instances or a minimum area coverage to ensure adequate representation of consolidated rock particles for reliable geological analysis.

The workflow 700 may accommodate different threshold values depending on the specific analysis requirements and the characteristics of the geological formations being investigated. The threshold criteria may be adjusted based on factors such as the expected particle size distributions, the degree of formation consolidation, and the specific geological information being sought from the analysis. The flexible threshold approach may enable optimization of the analysis process for different drilling environments and geological conditions while maintaining quality control standards for reliable results.

If the cutting threshold at 741 is not met, the patches may be discharged at block 739. If the threshold is met, the cuttings may be analyzed along a cutting analysis pipeline at block 737, producing cuttings results at block 735. Likewise, if the patches threshold at 749 is not met, the patches may all be discharged at 747. If the threshold is met, the patches at block 759 may proceed to be analyzed along a separate patches analysis pipeline at block 745, producing results for the patches at block 743. In this way, using the workflow 700 shown in FIG. 7, the consolidated cuttings and unconsolidated, loose material from a single sample of drilling cuttings can be segmented/identified, and analyzed along parallel processing paths to extract information regarding the geological formation from which they were extracted. The patches results for the unconsolidated fraction of the photographs of the sample may be combined with the cutting results for the consolidated rocks in the sample to produce an integrated set of results at block 733. This process may be repeated for the same or other cuttings samples as shown at block 769, where the workflow 700 begins again.

The parallel processing architecture implemented in the workflow 700 may ensure that both consolidated and unconsolidated material are processed using appropriate analytical techniques while maintaining coordination between the processing paths to enable meaningful integration of results. The workflow 700 may accommodate samples containing varying proportions of consolidated and unconsolidated material, automatically adjusting the analysis emphasis based on the material types present in each sample. The integrated results produced by the workflow 700 may provide comprehensive geological characterization that enables more accurate lithological classification, formation property estimation, and geological interpretation than conventional methods that focus solely on consolidated rock particles.

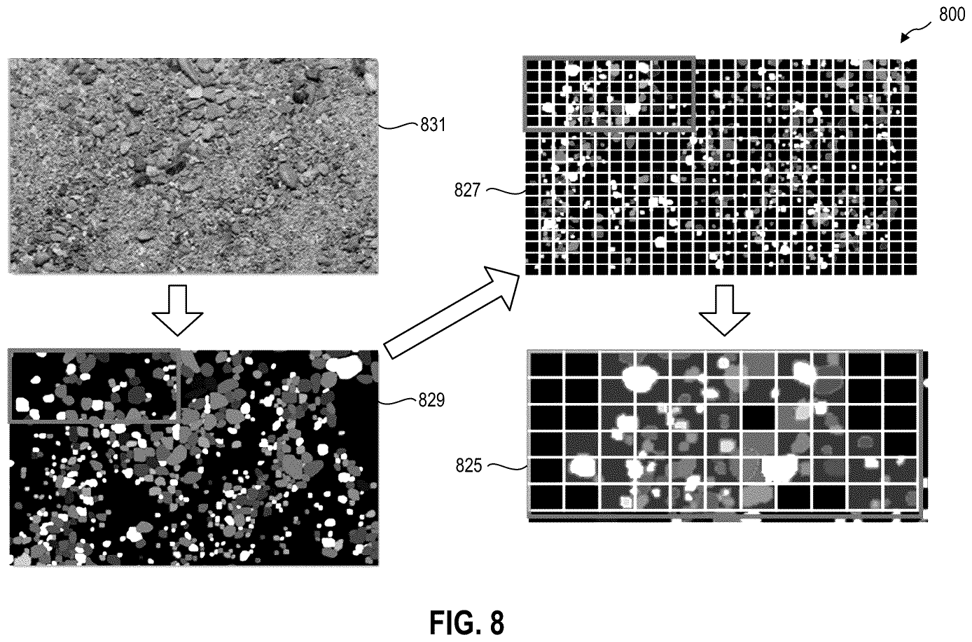

Referring to FIG. 8, a workflow 800 may illustrate a schematic representation of the transformation process from initial photograph acquisition to final segmentation and partitioning of drilling cuttings samples. The workflow 800 may demonstrate the sequential steps involved in processing a white-light photograph to identify consolidated rock particles through segmentation while simultaneously extracting unconsolidated material through systematic partitioning. The workflow 800 may provide a visual representation of how different material types within the same sample are processed using complementary image analysis techniques.

The workflow 800 may begin with a photograph 831 that represents a white-light image of a drilling cutting sample containing both consolidated rock particles and unconsolidated material. The photograph 831 may show the sample as captured under visible white light illumination, displaying the natural colors and textures of all materials present in the sample without any processing or analysis applied. The photograph 831 may serve as the input data for subsequent segmentation and partitioning processes that will separate and identify different material types within the sample.

At block 829, the workflow 800 may proceed to a segmentation process that identifies consolidated rock particles within the photograph 831 while treating the loose fraction present in the sample as background. The segmentation process may utilize instance segmentation algorithms to distinguish individual rock particles from the surrounding areas that contain unconsolidated material. During this process, the loose fraction may be classified as background and excluded from the segmented instances, allowing the segmentation algorithms to focus specifically on identifying consolidated rock particles that maintain their structural integrity.

The segmentation process may produce a representation where consolidated rock particles are clearly delineated and identified as individual instances, while areas containing unconsolidated material appear as background regions. The loose fraction present in the sample may be represented in black color in the segmented output, indicating that these areas have been classified as background rather than as segmented objects. This treatment of unconsolidated material as background may enable the segmentation algorithms to focus on consolidated particles while preserving the spatial information about loose material locations for subsequent partitioning processes.

At block 827, the workflow 800 may implement a partitioning process that divides the photograph 831 into a systematic grid pattern of uniform patches. The partitioning process may create a regular array of rectangular or square patches that cover the entire photograph area, enabling systematic analysis of unconsolidated material present in areas between the segmented rock particles. The grid pattern may be applied with no overlapping between adjacent patches, creating discrete analysis units that can be individually processed to extract geological information from loose material.

The partitioning process may generate patches that are systematically distributed across the entire photograph 831, regardless of the material types present in each patch location. However, the workflow 800 may subsequently evaluate each patch to determine whether the patch contains primarily unconsolidated material or overlaps with segmented consolidated rock particles. Patches that overlap with segmented cuttings may be identified and excluded from further processing to prevent analysis of mixed material types within individual patches.

At block 825, the workflow 800 may implement a filtering mechanism that excludes patches containing segmented rock particles or sample trail background from subsequent analysis processes. Patches depicted may represent areas that are excluded from next workflow steps due to overlap with consolidated particles or presence of background material that does not contain geological information. The filtering process may ensure that only patches containing primarily unconsolidated material are retained for analysis, preventing contamination of loose material analysis with data from consolidated particles or non-geological background areas.

The exclusion of overlapping patches may be performed by comparing the spatial locations of the segmented rock particles with the positions of the partitioned patches within the photograph 831. Patches that contain pixels belonging to segmented cuttings may be automatically identified and marked for exclusion from the unconsolidated material analysis pipeline. Similarly, patches that contain significant portions of sample trail background or other non-geological areas may be excluded to ensure that subsequent analysis focuses on patches containing meaningful geological material.

The workflow 800 may demonstrate how the hybrid segmentation approach enables comprehensive analysis of both consolidated and unconsolidated material types present in non-sieved drilling cuttings samples. The segmentation process may preserve the natural shapes and boundaries of consolidated rock particles while the partitioning process may create uniform analysis units for processing loose material. The combination of these complementary approaches may enable extraction of geological information from both material types without loss of data that would occur in conventional sieving processes.

The schematic representation provided by the workflow 800 may illustrate the spatial relationship between segmented rock particles and partitioned patches within the same photograph 831. The visual representation may show how areas containing consolidated particles are processed through instance segmentation while areas containing unconsolidated material are processed through patch-based analysis. The workflow 800 may ensure that each portion of the photograph 831 is assigned to the most appropriate analysis pathway based on the material type present in that region, enabling comprehensive geological characterization of the complete drilling cuttings sample.

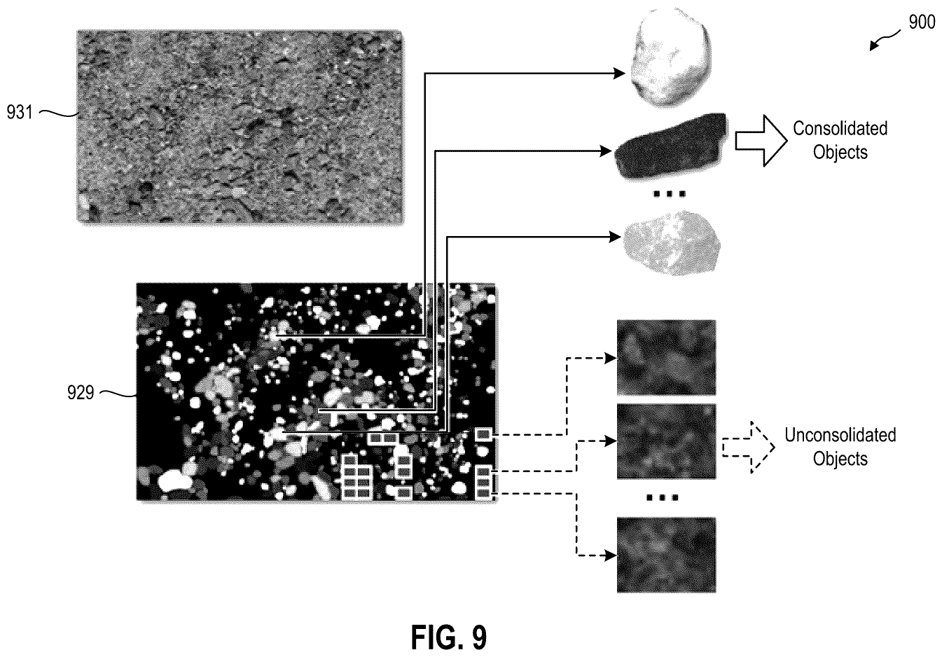

Referring to FIG. 9, a workflow 900 may illustrate a schematic representation of extracted objects that result from the hybrid segmentation and partitioning processes applied to drilling cuttings samples. The workflow 900 may demonstrate how different material types present in the same sample are processed and represented as distinct object categories that can be analyzed using appropriate techniques for each material type. A photograph 931 may show the spatial arrangement of both consolidated and unconsolidated materials as they appear in the original drilling cuttings sample before processing.

At block 929, the workflow 900 may show consolidated objects that represent individual rock particles extracted through instance segmentation processes. The consolidated objects may maintain their natural irregular shapes and varying sizes as determined by the geological processes that formed the original rock, and the mechanical forces encountered during drilling operations. Each consolidated object may correspond to a rock particle that has retained its structural integrity during the drilling process and can be analyzed as an individual geological specimen.

The consolidated objects may exhibit diverse morphological characteristics that reflect the lithological properties of the subterranean formation from which the drilling cuttings sample was extracted. The consolidated particles may include rock particles from a poorly cemented lithology, where some portions of the formation maintain sufficient structural integrity to produce intact rock fragments during drilling operations. The consolidated objects may belong to different lithologies such as sandstone, siltstone, or other rock types that may be present within the same geological formation or drilling interval.

The workflow 900 may also show unconsolidated objects that represent uniform square patches extracted through the partitioning process applied to areas containing loose material. The unconsolidated objects may be represented as standardized rectangular or square analysis units that enable systematic processing of disaggregated mineral components and loose grains present in the drilling cuttings sample. Each unconsolidated object may correspond to a patch that contains primarily loose material without significant overlap with consolidated rock particles.

The unconsolidated material may include loose sand from a poorly cemented lithology, where portions of the formation have become disaggregated during drilling operations due to weak cementation or structural instability. The unconsolidated loose material may include disaggregated components from a poorly cemented lithology that have separated into individual grains, crystals, or other fine-grained components during the drilling process. The disaggregated components from the poorly cemented lithology may include loose sand from poorly consolidated sandstone that comes to the surface in the form of loose material rather than intact rock fragments or from carbonate (e.g., grainstones).

The unconsolidated material patches may be classified and associated as sand when drilling in clastic formations, particularly when the drilling operations encounter poorly consolidated sandstone formations. The classification approach may enable systematic categorization of loose material based on the geological context and formation type being drilled. When drilling in clastic formations, the unconsolidated objects may be assigned to a sand lithology classification, reflecting the disaggregated nature of the original sandstone formation.

The workflow 900 may demonstrate how the hybrid analysis approach enables extraction of geological information from both consolidated and unconsolidated fractions of the same drilling cuttings sample. The consolidated objects may provide information about the structural and mineralogical properties of intact rock portions, while the unconsolidated objects may provide information about the grain size distributions, mineral compositions, and textural characteristics of the disaggregated components. The combination of both object types may enable comprehensive geological characterization that would not be possible using conventional analysis methods that focus solely on consolidated particles.

The distinction between consolidated and unconsolidated objects may reflect the varying degrees of cementation and structural integrity present within the same geological formation. A poorly cemented lithology may produce both consolidated particles that maintain their structural integrity and unconsolidated material that becomes disaggregated during drilling operations. The poorly cemented lithology may include sandstone and/or carbonate formations where some portions remain sufficiently cemented to produce intact rock fragments while other portions disaggregate into loose sand components.

The processing pathways for consolidated and unconsolidated objects may utilize different analytical techniques optimized for the characteristics of each material type. The consolidated objects may be processed using methods appropriate for intact rock particles, such as shape measurements, morphological analysis, and structural characterization. The unconsolidated objects may be processed using methods appropriate for loose material, such as grain size distribution analysis, texture analysis of individual grains, and compositional analysis of disaggregated mineral components.

The workflows, methods, and systems described herein may enable comprehensive geological analysis by preserving and utilizing information from both material types that may be present in drilling cuttings samples obtained from poorly cemented lithologies. The approach may prevent loss of geological information that would occur if unconsolidated material were discarded during conventional sieving processes. The integrated analysis of both consolidated and unconsolidated objects may provide enhanced understanding of the geological formation properties, and the degree of cementation present within the drilled interval.

The analysis of unconsolidated material patches through a first processing pipeline may enable extraction of comprehensive geological information from disaggregated components present in drilling cuttings samples. The first processing pipeline may be specifically designed to process loose material characteristics and may implement multiple analytical techniques that are optimized for analyzing grain-scale properties of unconsolidated material. The first processing pipeline may analyze a subset of patches that have been extracted from areas containing primarily loose grains, disaggregated crystals, and other fine-grained components that result from poorly cemented lithologies during drilling operations.

Spectral measurements may be performed on the subset of patches to characterize the spectral properties of individual mineral grains and disaggregated components present in the unconsolidated material. The spectral measurements may utilize RGB channel data obtained from both visible light and UV fluorescence photographs to identify mineral compositions and detect variations in mineralogical content within the loose material fraction. The spectral measurements may enable identification of specific mineral types based on their characteristic colors and fluorescence responses, providing information about the mineralogical composition of the original formation before disaggregation occurred during drilling operations.

Texture analysis may be conducted on the subset of patches to characterize the surface properties and structural characteristics of the disaggregated components within the unconsolidated material. The texture analysis may examine patterns, roughness variations, and grain surface features that can indicate the original rock texture and the degree of weathering or alteration that may have contributed to the disaggregation process. The texture analysis may provide information about the depositional environment and diagenetic processes that affected the formation, as well as the mechanical properties that influenced the disaggregation behavior during drilling operations.

Grain size distribution analysis may be performed on the subset of patches to determine the size characteristics and sorting properties of the loose material components. The grain size distribution analysis may measure individual grain dimensions within each patch and compile statistical distributions that characterize the overall size properties of the unconsolidated material. The grain size distribution analysis may provide information about the depositional processes that formed the original sedimentary formation and may enable correlation with established geological classification systems for sedimentary rocks and their disaggregated equivalents.

Reservoir parameter estimation may be conducted using the analytical results obtained from the subset of patches to predict formation properties that would be measured by conventional tools. The reservoir parameters may com from logs, laboratory measurements (XRF, XRD, SCAL, CCAL . . . etc.), or visual observation. In various examples, each of these reservoir parameter data can be the result of a different method that applies to the image objects extracted in the methods described herein (e.g., patches and instances). The reservoir parameter estimation may correlate the spectral measurements, texture analysis results, and grain size distribution data with known relationships between these properties and formation characteristics such as porosity, permeability, and lithological composition. The reservoir parameter estimation may enable prediction of formation properties in intervals where conventional logging data may not be available, providing enhanced geological characterization of the subterranean formation from the unconsolidated loose material that would otherwise be discarded in conventional analysis workflows.

The analysis of consolidated rock particles through a second processing pipeline may enable extraction of comprehensive geological information from intact rock fragments present in drilling cuttings samples. The second processing pipeline may be specifically designed to process the characteristics of consolidated particles and may implement multiple analytical techniques that are optimized for analyzing the structural and compositional properties of rock particles that have maintained their integrity during drilling operations. The second processing pipeline may analyze segmented instances that have been identified through instance segmentation processes, where each segmented instance represents an individual consolidated rock particle with defined boundaries and morphological characteristics.

Shape measurements may be performed on the segmented instances to quantify the geometric characteristics and morphological properties of the consolidated rock particles. The shape measurements may determine particle dimensions, aspect ratios, circularity indices, and other geometric parameters that characterize the three-dimensional form of each rock particle. The shape measurements may provide information about the original rock texture, the degree of particle fragmentation that occurred during drilling operations, and the mechanical properties of the formation that influenced particle breakage patterns. The shape analysis may enable identification of different rock types based on their characteristic particle morphologies and may provide insights into the structural competence of the geological formation from which the particles were extracted.

Texture measurements may be conducted on the segmented instances to characterize the surface properties and internal structural variations present within the consolidated rock particles. The texture measurements may analyze surface roughness, grain boundary patterns, mineral distribution heterogeneity, and other textural features that reflect the depositional environment and diagenetic history of the original rock formation. The texture measurements may utilize both visible light and UV fluorescence image data to enhance the detection of subtle textural variations that may not be apparent under single illumination conditions. The texture analysis may provide information about the depositional processes, metamorphic grade, and alteration history that affected the formation, enabling more accurate lithological classification and geological interpretation.

Spectral measurements may be performed on the segmented instances using spectral data obtained from both visible light and UV fluorescence photographs to identify mineral compositions and detect lithological variations within the consolidated rock particles. The spectral measurements may analyze RGB channel data from multiple illumination conditions to enhance mineral differentiation and enable identification of specific mineral assemblages present within each rock particle. The color analysis may utilize the fluorescent properties revealed under UV illumination to distinguish between minerals that may appear similar under visible light alone, providing enhanced accuracy in mineralogical identification and compositional analysis.

Feature extraction may be conducted on the segmented instances to identify and quantify specific geological characteristics that can be used for lithological classification and formation property estimation. The feature extraction may utilize data from multiple RGB channels obtained from both visible light and UV illuminated photographs to maximize the geological information available for analysis and classification processes. The feature extraction may identify diagnostic features such as mineral grain sizes, crystal habits, weathering patterns, and other geological indicators that characterize the consolidated rock particles. The combined results from shape measurements, texture measurements, spectral measurements, and feature extraction may provide comprehensive geological characterization of the consolidated particles, enabling accurate lithological classification and estimation of formation properties such as rock strength, porosity characteristics, and depositional environment conditions.

The methods of drilling cuttings analysis described herein have been primarily described with reference to wellbore drilling operations. The methods described herein may be used in applications other than in the context of drilling operations for a wellbore. In other embodiments, methods according to the present disclosure may be used outside a wellbore or other downhole environment used for the exploration or production of natural resources.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers'specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

1. A method for analyzing drilling cuttings, comprising:
extracting a sample of the drilling cuttings from a subterranean formation, the sample comprising consolidated particles and unconsolidated material;
photographing the sample to produce a photograph;
performing an image analysis on the photograph to identify segments of the photograph visualizing the unconsolidated material and excluding visualization of the consolidated particles; and
analyzing the segments by performing at least one of a spectral measurement, a texture analysis, a grain size distribution analysis, or a reservoir parameter estimation of the segments.

2. The method of claim 1, wherein:
the segments are first segments; and
performing the image analysis includes identifying second segments of the photograph visualizing the consolidated particles.

3. The method of claim 2, further comprising analyzing the second segments.

4. The method of claim 3, wherein:
the spectral measurement is a first spectral measurement; and
analyzing the second segments comprises performing at least one of a shape measurement, a texture measurement, a second spectral measurement, or a feature extraction of the second segments.

5. The method of claim 1, wherein extracting the sample from the subterranean formation comprises drilling into a poorly cemented lithology.

6. The method of claim 5, wherein the poorly cemented lithology comprises at least one of sandstone or carbonate.

7. The method of claim 6, wherein the unconsolidated material comprises loose sand from the sandstone.

8. A method for analyzing drilling cuttings, comprising:
extracting, from a geological formation, a sample of non-sieved drilling cuttings comprising consolidated rock particles and unconsolidated loose material;
acquiring, via a camera, a photograph of the sample;
performing image segmentation on the photograph to identify the consolidated rock particles as segmented instances;
partitioning the photograph into a plurality of patches;
extracting a subset of patches of the plurality of patches, the subset of patches being non-overlapping with the segmented instances;
analyzing the subset of patches via a first processing pipeline to obtain first geological information of the geological formation from the unconsolidated loose material; and
analyzing the segmented instances through a second processing pipeline to obtain second geological information of the geological formation from the consolidated rock particles.

9. The method of claim 8, wherein each patch of the plurality of patches is non-overlapping with adjacent patches of the plurality of patches.

10. The method of claim 8, further comprising:
applying a first minimum threshold test to the subset of patches based on a first total number of individual patches of the subset of patches or a first area of the photograph covered by the subset of patches; and
applying a second minimum threshold test to the segmented instances based on a second total number of the segmented instances or a second area of the photograph covered by the segmented instances.

11. The method of claim 10, further comprising:
discharging the subset of patches that fall below the first minimum threshold; and
discharging the segmented instances that fall below the second minimum threshold.

12. The method of claim 8, wherein analyzing the subset of patches comprises performing at least one of a first spectral measurement, a texture analysis, a grain size distribution analysis, or a reservoir parameter estimation.

13. The method of claim 12, wherein analyzing the segmented instances comprises performing at least one of a shape measurement, a texture measurement, a second spectral measurement, or a feature extraction.

14. The method of claim 8, wherein the unconsolidated loose material comprises disaggregated components from a poorly cemented lithology.

15. The method of claim 14, wherein the disaggregated components from the poorly cemented lithology includes at least one of loose sand from poorly consolidated sandstone or carbonate.

16. The method of claim 8, wherein acquiring the photograph comprises taking a first photograph under visible white light illumination and a second photograph under UV light illumination.

17. The method of claim 16, wherein the UV light illumination comprises at least one of short UV light illumination and long UV light illumination to produce a plurality of fluorescence colors in minerals present in the sample.

18. The method of claim 8, wherein the photograph is acquired when the sample is on a tray having a contrast color comprising at least one of magenta, blue, or green.

19. A method for analyzing a geological formation, the method comprising:
extracting a sample of drilling cuttings from the geological formation, the sample comprising consolidated particles and unconsolidated material;
photographing the sample to produce a photograph;
performing an image analysis on the photograph to identify first segments of the photograph visualizing the unconsolidated material and second segments of the photograph visualizing the consolidated particles;
analyzing the first segments to obtain first information of the geological formation by performing at least one of a first spectral measurement, a texture analysis, a grain size distribution analysis, or a reservoir parameter estimation of the first segments; and
analyzing the second segments to obtain second information of the geological formation by performing at least one of a shape measurement, a texture measurement, a second spectral measurement, or a feature extraction of the second segments.

20. The method of claim 19, wherein:
the geological formation comprises a poorly cemented lithology;
the unconsolidated material comprises loose sand from the poorly cemented lithology; and
the consolidated particles comprise rock particles from the poorly cemented lithology.
